(19) United States
(12) Patent Application Publication
(10) Pub. No.: US 2005/0101938 A1
(43) Pub. Date: May 12, 2005

(54) Guidewire for use in colonic irrigation

(76) Inventor: Arnold R. Leiboff, Stony Brook, NY (US)

Correspondence Address: STEINBERG & RASKIN, P.C., 1140 AVENUE OF THE AMERICAS, 15th FLOOR, NEW YORK, NY, 10036-5803, US

(21) Appl. No.: 10/702,303

(22) Filed: Nov. 6, 2003

(51) Int. Cl. A61M 25/01

(52) U.S. Cl. 604/528

(57) ABSTRACT

Guidewire for use in body cavity irrigation devices includes an elongate, resilient shaft adapted to pass in an interior of or along an irrigating tube and a bulbous enlargement arranged at a distal end of the shaft. The bulbous enlargement has a smooth, arcuate outer surface to prevent perforation of the walls of the body cavities in which it moves. For colonic irrigation, the guidewire is inserted into the colon and then the irrigating tube is slid over or along the guidewire. Fluid is directed through the irrigating tube to the treatment site or sites in the colon.

### FIELD OF THE INVENTION

The invention relates generally to devices and methods for irrigating and draining an internal body cavity and more particularly to guidewires for guiding and facilitating passage of tubular devices through the colon and methods of using a guidewire and tubular devices for colonic irrigation and postoperative colonic drainage.

### BACKGROUND OF THE INVENTION

Surgical operations on the colon and rectum are frequently required to treat diseases such as colorectal neoplasia, diverticulitis, and inflammatory bowel disease. Surgeons believe that it is easier and safer to operate on the colon and rectum if these organs are first cleansed of feces. Most of the time cleansing is achieved prior to the operation through the ingestion of cathartics such as phospho-soda or polyethelene glycol solutions. In circumstances which preclude preoperative bowel preparation, intraoperative bowel cleansing is sometimes performed. Fluid can be introduced into the rectum or colon during the surgical operation. The fluid is then drained along with suspended and particulate stool. Apparatus, such as those described in U.S. Pat. No. 4,637,814 discussed in detail below, have been developed to facilitate intraoperative bowel cleansing. Such systems prevent contamination of the sterile operative field in order to prevent postoperative infections.

One way to cleanse the bowel is to attach a drain tube to the distal end of the bowel segment to be cleansed, introduce lavage fluid through the drain tube, and drain the bowel through the same drain tube. Alternatively, a second tube can be placed into the bowel through an enterotomy at the proximal end of the segment to be cleansed, and fluid introduced through this proximal tube. The fluid then traverses the bowel segment and drains out through the drain at the distal end. Lavage by these methods is usually time consuming and often incomplete. It takes time for the fluid to fill and traverse the bowel, and time to drain. Filling the bowel by infusing fluid into one end, causes dilitation of the bowel at the point of entry. Fluid is then slowly distributed through the bowel by hydrostatic pressure, gravity, peristalsis, and manual external pressure (“milking the bowel”). These same forces cause the fluid and feces to drain out.

U.S. Pat. No. 4,637,814 describes an apparatus and method which facilitates intraoperative bowel cleansing. An irrigating tube is passed into and through much or all of the bowel segment which is to be cleansed. Filling the bowel with lavage fluid may be easier and more rapid because the irrigating tube ensures a large conduit for dispersion of the fluid and prevents segmentation of the bowel by muscular constrictions. Emptying the bowel of lavage fluid and suspended feces can be facilitated by applying suction to the irrigating tube and aspirating the bowel contents through the tube. When the tube has multiple apertures, irrigation and aspiration are more efficient.

However, the use of an irrigating tube is problematic in that the tube is typically flexible and as such, is difficult, if not impossible, to push through a long segment of bowel.

Therefore, a colonic irrigation device was developed for intraoperative bowel cleansing which includes a guidewire adapted to facilitate passage of an irrigating tube through the bowel. The guidewire is introduced first into the bowel, either through the anus or through an open end of the colon. The guidewire is advanced until its tip is positioned at the most proximal extent of the bowel to be lavaged. The irrigating tube is then slid into position over the guidewire. When in position, water, saline or other treatment fluid is directed through the irrigating tube to cleanse the colon.

On rare occasions, guidewires are employed for the purpose of positioning tubes or stents within the colon, but usually in conjunction with a colonoscope, for example in the treatment of colonic ileus. Colonic ileus (Oglvies Syndrome) is a condition where the colon has temporarily lost its power to contract. The colon remains dilated and forms a functional obstruction to the passage of feces and gas. To treat this condition, a colonoscope may be inserted through the colon, and the colon is aspirated and decompressed. Through a lumen of the colonoscope, a guidewire may be passed, and the colonoscope may be removed while the guidewire remains in position, and a tube then passed over the guidewire. The tube is then secured in place and left to keep the colon decompressed. However, such a guidewire cannot be easily passed through the bowel without the use of a colonscope. When passed into the bowel on its own, its straight tip gets caught against the bowel wall and the pliable shaft bends instead of advancing the tip. Folds in the bowel wall formed by the curvature of the bowel and by the contraction of its circular muscle aggravate the problem of passing a conventional guide wire. Making the shaft more rigid does not make passage much easier, but makes it more likely that the bowel will be inadvertently injured or perforated. These same problems arise when passing conventional guidewires through the bowel during a laparotomy, when the bowel can be directly manipulated.

Not only does the straight narrow tip of conventional guidewires hinder passage through bowel for reasons stated above, but the narrow tip also makes the tip difficult to feel through the bowel wall and nearly impossible to grasp. The tip, therefore, cannot be manipulated by the surgeon to aid in passage of the guidewire through the bowel.

In addition to use in colonic irrigation devices, guidewires are also used in other invasive body cavity examinations. For example, U.S. Pat. No. 5,746,692 describes a method for using a resilient member to guide a catheter to an observation site in an interior body passage and arranging an endoscope in the catheter and to be movable through the catheter body to a position in which the observation site can be viewed. The resilient member extends beyond the distal end of the catheter body and is designed to displace the object being viewed from the distal end of the catheter body to enable the endoscope to properly view the observation site. The resilient member includes a wire encased in a sheath and a rounded enlargement at a terminal distal end. The maximum cross-sectional dimension in a radial plane of the rounded enlargement is between 0.5 millimeters and 1.2 millimeters.

The observation device of the '692 patent, in which the endoscope is passed through the catheter body the distal end of which is spaced from the observation site by the resilient displacement member, is fundamentally different from body cavity irrigation devices in which fluid is passed through an irrigating tube extending over and along a guidewire to a treatment site in the body cavity. Moreover, the rounded enlargement in the device of the '692 patent is of such small size that it would not be easily palpable and graspable through the bowel wall and thus not very helpful to a surgeon who is trying to pass a device through the colon.

It would thus be desirable to construct a guidewire for use in bowel irrigation which enables a surgeon to easily discern the tip and manipulate the guidewire through the bowel wall thereby enabling him to guide and maneuver the guidewire through the bowel.

### OBJECTS AND SUMMARY OF THE INVENTION

It is an object of the invention to provide a new and improved guidewire for use in devices and methods for irrigating an internal body cavity, such as colonic irrigation devices and methods.

It is another object of the invention to provide new and improved devices and methods for irrigating an internal body cavity and more particularly to colonic irrigation devices and methods.

It is another object of the invention to provide new and improved colonic irrigation devices including a guidewire for guiding and maneuvering an irrigating tube into the colon and a method of using such a colonic irrigation device.

It is yet another object of the present invention to provide new and improved colonic irrigation devices including a guidewire having a tip which is discernible and graspable from an exterior of the colon to thereby enable the guidewire to be guided and manipulated by a surgeon performing the colonic irrigation procedure.

It is another object of the invention to provide new and improved colonic irrigation devices including a guidewire having a tip which will not perforate a bowel wall during movement of the guidewire into and through the bowel.

In order to achieve these objects and others, a guidewire in accordance with the invention includes an elongate, resilient shaft adapted to pass in an interior of or along an irrigating tube and a bulbous enlargement arranged at a distal end of the shaft. The bulbous enlargement has a smooth, arcuate outer surface to prevent perforation of the walls of the body cavities, particularly the bowel wall, in which it moves.

The bulbous enlargement constitutes a large round tip at the end of the guide wire which helps the surgeon manipulate the guide wire through the bowel in numerous identifiable ways:
1. It allows the tip of the guide wire to be easily located through the bowel wall.
2. It allows the surgeon to more easily manipulate the tip through the bowel wall, redirecting the tip.
3. It allows the tip to more easily slide forward against the bowel wall when being advanced, particularly when the surgeon supports the wall of the bowel with his hand, when the guidewire is pushed from below.
4. In an area of bowel constricted by muscular contraction, it can dilate the bowel and thereby flatten folds and facilitate passage of the guidewire through the bowel.
5. It is less likely to perforate the bowel wall because of its roundness and larger surface area.
6. It can be grasped by the surgeon together with the bowel wall while the guidewire is partially withdrawn. This accordians the bowel on the shaft of the guidewire, effectively shortening the length of the bowel. The guide wire can then be advanced while holding the distal bowel and preventing it from uncrimping. This prevents elongation and bowing of the bowel and facilitates passage of the guide wire through the bowel.
7. When the tip of the guidewire has been advanced to the most proximal part of the colon to be irrigated, the bowel and tip can again be grasped together as the guidewire is withdrawn, to shorten the distance for insertion of the irrigating tube, as described below.

In use for irrigating a treatment site in the bowel, the guidewire is inserted into the bowel and then the irrigating tube is slid over or along the guidewire. Fluid is directed through the irrigating tube to the treatment site or sites in the bowel. For example, the guidewire may be pushed through the anus and rectum into and through the colon, with assistance from the surgeon manipulating the guidewire through the bowel wall, and then the irrigating tube is passed over or along the guidewire and fluid directed therethrough to cleanse or irrigate the colon. Alternatively, the guidewire can be introduced into the colon through a drain tube which is attached to the distal end of the segment of colon to be irrigated.

The size of the bulbous enlargement is an important feature of the invention since it is desirable to enable the bulbous enlargement to be discernible, grasped and manipulated through the wall of the bowel. Thus, the bulbous enlargement may be spherical and have a diameter from about 0.25 inches to about 0.75 inches.

A colonic irrigation device in accordance with the invention thus includes a guidewire having a bulbous enlargement at a distal end and which is adapted to be guided into a colon, an irrigating tube movable relative to and over or along the guidewire and a system for providing a flow of fluid through the irrigating tube. In use, the guidewire is guided first into the colon and then the irrigating tube is moved over or along the guidewire into the colon. Once the irrigating tube is in place, fluid is directed therethrough directly to the treatment site. If the diameter of the bulbous enlargement is smaller than the internal diameter of the irrigating tube, the guidewire can be removed before irrigation.

### BRIEF DESCRIPTION OF THE DRAWINGS

The invention, together with further objects and advantages thereof, may best be understood by reference to the following description taken in conjunction with the accompanying drawings wherein like reference numerals identify like elements.

FIG. 1 illustrates in cross-section a rectum and colon of a human being and a detail of a colonic irrigation device in accordance with the invention including a first embodiment of a guidewire in accordance with the invention.

FIG. 2 is a cross-section taken along the line 2-2 in FIG. 1.

FIG. 3 is a front view of the first embodiment of a guidewire in accordance with the invention.

FIG. 4 is a front view of a second embodiment of a guidewire in accordance with the invention.

FIG. 5 is a front view of a third embodiment of a guidewire in accordance with the invention.

### DETAILED DESCRIPTION OF THE INVENTION

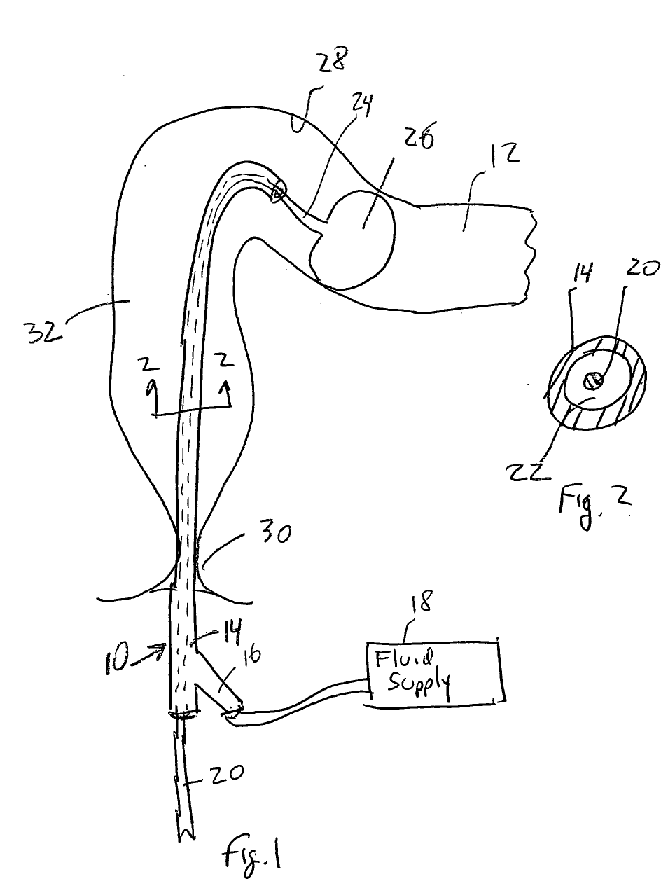

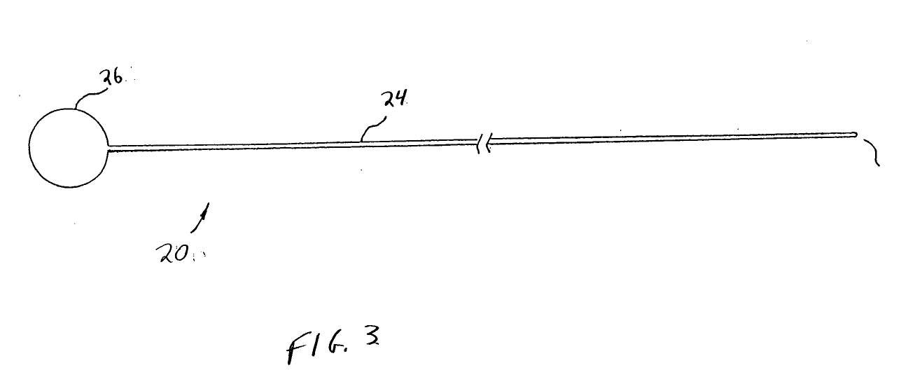

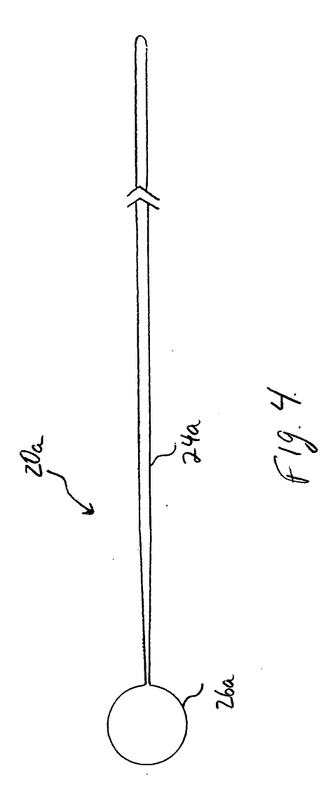

Referring to the accompanying drawings wherein like reference numerals refer to the same or similar elements, FIG. 1 shows an irrigation device 10 for irrigating the colon 12. The irrigation device 10 generally includes an irrigating tube 14 having a connector 16 for connecting the tube 14 to a fluid supply 18, and a guidewire 20 passing through an interior of the tube 14 (see FIG. 2). The tube 14 and guidewire 20 are movable relative to one another. In use, fluid flows from the fluid supply 18 through the connector 16 into the tube 14 and then through the tube 14, in the space 22 defined between the tube 14 and the guidewire 20, to the distal end of the tube 14, which is placed in proximity to or at a treatment site.

It should be understood that the use of the guidewire 20 in connection with the irrigating tube 14 in the manner shown in FIG. 1 is one exemplifying use and that there are other uses of the guidewire 20 in accordance with the invention. For example, another exemplifying use of the guidewire 20 is in a case where a drain tube, such as disclosed in U.S. Pat. No. 4,637,814 and U.S. Pat. No. 5,443,445 (incorporated by reference herein), is attached to a distal end of a bowel segment to be cleansed. In this case, the drain tube is inserted into the bowel segment and attached thereto and an irrigating tube is passed through the drain tube into and through at least a portion of the bowel segment to be cleansed. Lavage fluid is introduced into the bowel through the irrigating tube and is removed from the bowel segment through the drain tube. When the guidewire 20 in accordance with the invention is used for this arrangement, it is inserted through the drain tube, for example to the most proximal extent of the bowel, before the irrigating tube is inserted into the bowel and thereafter the irrigating tube is slid over the guidewire 20 into the bowel. It has been found that it is easier and quicker to pass and position both the guidewire 20 and irrigating tube in the bowel than it is to pass and position an irrigating tube without using the guidewire 20.

In accordance with one embodiment of the invention, the guidewire 20 includes an elongated, resilient wire or shaft 24 formed of metal or plastic (e.g., TEFLON(™)) optionally encased in a sheath and a bulbous enlargement 26 attached at a terminal distal end of the shaft 24. If formed of metal, the shaft 24 may be formed from a metal such as Nitinol™ which has superior memory properties.

The bulbous enlargement 26 is attached to the shaft and has a smooth, arcuate outer surface to enable the bulbous enlargement 26 to slide along the bowel walls 28. The bulbous enlargement 26 is, like the shaft 24 and the tube 14, made of a material compatible for insertion into and contact with internal body cavities, such materials being known to those skilled in the art. Also, the bulbous enlargement 26 should be sufficiently rigid to enable it to be easily guided to the colon 12, although the shaft 24 is flexible.

The design of the guidewire 20 allows it to be passed through the bowel much easier and quicker than an irrigating tube since the rounded outer surface of the bulbous enlargement 26 slides along the bowel wall 28 when making contact therewith. Furthermore, the guidewire 20 is stiffer than the irrigating tube, which facilitates advancement through the bowel, and its shaft 24 is thinner and has a lower coefficient of friction than the irrigating tube, reducing the resistence to passage through a tortuous bowel.

The bulbous enlargement 26 may be generally spherical, as shown, and have a diameter of about 0.25 inches to about 0.75 inches. A more specific range would be from about 0.5 inches to about 0.75 inches.

Instead of being spherical as shown, the bulbous enlargement 26 may have other configurations which provided smooth arcuate outer surfaces. For example, only the forward-facing outer surface of the bulbous enlargement 26 can be arcuate and smooth (hemispherical is a possibility), while the rearward-facing outer surface can be other than hemispherical since the rearward-facing outer surface is not usually press against a bowel wall and thus the danger of perforating a bowel wall is less. The edges of the bulbous enlargement 26 should however be smooth and arcuate to prevent perforation of the bowel wall upon withdrawal of the guidewire 20 from the colon 12.

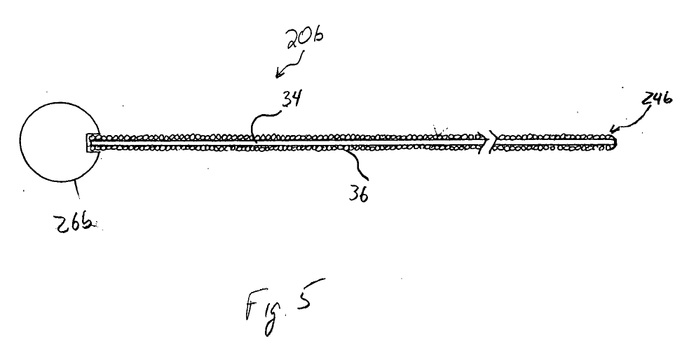

FIG. 4 shows a second embodiment of the guidewire, designated 20a, in which the shaft 24a is tapered so that its diameter in proximity to the bulbous enlargement 26a is smaller than the diameter farther from the bulbous enlargement 26a. This gives the guidewire 20a more flexibility near the bulbous enlargement 26a, facilitating manipulation of the bulbous enlargement 26a and advancement through the bowel lumen.

FIG. 5 shows a third embodiment of the guidewire 20b in which the shaft 24b is constructed of a straight or tapering central metal core wire 34 surrounded by a wire coil 36. This configuration increases the diameter of the shaft 24b while maintaining flexibility. The larger diameter of the shaft 24b allows for easier and safer handling of the guidewire 20b and makes it less likely to injure the wall of the bowel.

It is also conceivable to make the shaft 24 of the guidewire 20 such that a portion thereof attached to the bulbous enlargement 26 is more flexible than the remainder of the shaft 24. For example, the flexibility of a segment of metal or plastic can be increased without altering its diameter. The shaft 24 would thus have a common diameter yet different flexibility along its length.

In use for irrigating treatment sites in the colon 12 in the manner shown in FIG. 1, the bulbous enlargement 26 is first pushed through the anus 30 into the rectum 32. The guidewire 20 is then grasped and pushed through the anus 30, so that the bulbous enlargement 26 progresses through the rectum 32. If the bulbous enlargement 26 contacts any bowel wall 28, it slides along the bowel wall 28 in view of its smooth, arcuate forward-facing outer surface and therefore does not perforate the bowel wall 28. At the end of the rectum 32, the bulbous enlargement 26 is pushed into the colon 12 and then travels through the colon 12 as the guidewire 20 is pushed further inward. The surgeon, working within the abdominal cavity after gaining entry via the abdominal incision, grasps or manipulates the bulbous enlargement 26 through the intact bowel wall and helps advance the guidewire.

When the bulbous enlargement 26 has reached the most proximal extent of the colon 12 to be irrigated (most frequently the cecum), the irrigating tube 14 can then be fed over the guidewire 20 through the anus 30 into the rectum 32 and further into the colon 12 as shown in FIG. 1. The irrigating tube 14 is thus guided over the guidewire 20 through the colon 12. An alternative embodiment would have the irrigating tube 14 being guided along, but not over, the guidewire 20 to the most proximal part of the colon 12 to be irrigated.

Another advantage of the bulbous enlargement 26 is to enable the surgeon to grasp the bowel and guidewire tip together as the guidewire is partially withdrawn. This shortens and straightens the insertion path for the irrigating tube. When the proximal end of the irrigating tube reaches the bulbous enlargement, both irrigating tube and guidewire are advanced together to uncrimp the bowel. This maneuver can simplify and expedite the insertion of the irrigating tube.

The construction of a colonic irrigation device as shown in FIG. 1 is very general. Other types of bowel irrigation devices may also be used with a guidewire in accordance with the invention.

Another method of use of the guidewire 20 is in conjunction with a drain tube as disclosed in U.S. Pat. No. 4,637,814 and U.S. Pat. No. 5,443,445. In this method, after the drain tube is joined to the end of bowel, and after much of the stool has been drained from the bowel, a distal end of the drain tube is opened and the guidewire 20 is passed through the drain tube and bowel until the bulbous enlargement 26 lies in the preferred position within the bowel, which generally would be the cecum, the most proximal portion of the colon. The distal end of the guidewire 20 is then inserted through the opening in a proximal end of an irrigating tube and the irrigating tube is passed over the guidewire 20 until its proximal end sits adjacent to the bulbous enlargement 26 of the guidewire 20. The distal end or hub of the irrigating tube (or extensions attached thereto) is then attached to inflow and outflow tubing and irrigation of the bowel proceeds. Thus, the guidewire 20 may be present, with its shaft 24 extending through the irrigating tube 14, during the bowel cleaning procedure. However, if the bulbous enlargement 26 of the guidewire 20 is smaller in diameter than the lumen of the irrigating tube 14, then the guidewire 20 can be withdrawn through the irrigating tube 14 and removed prior to the attachment of extensions and prior to irrigation.

Alternatively, the guidewire 20 may be substantially longer than the irrigating tube, and prepared, packaged, and presented to the surgeon situated through the irrigating tube. The surgeon then advances the guidewire 20 through the bowel first. When the guidewire 20 is positioned in the bowel, its distal end may protrude from the distal end of the irrigating tube. The protruding guidewire 20 can then be grasped and the irrigating tube passed over the guidewire 20 into position in the bowel. Once the irrigating tube is in position, the guidewire 20 can be severed as it exits the distal end of the irrigating tube, so that tubing extensions can be attached to the irrigating tube. Or, if the bulbous enlargement 26 of the guidewire is smaller in diameter than the lumen of the irrigating tube 14, the guidewire 20 can be fully withdrawn through the irrigating tube 14 and removed before the extensions are attached to the irrigating tube 14.

In addition, the description above has been based on the use of the device for colonic irrigation. The same device, and method for using the same, are applicable to treatments and examinations in other body cavities, whether body cavities in humans or animals. Thus, the invention is not limited to colonic irrigation devices and methods and encompasses the any type of invasive body cavity examination in which it is desired to direct a fluid to a treatment site in a body cavity.

While particular embodiments of the invention have been shown and described, it will be obvious to those skilled in the art that changes and modifications may be made without departing from the invention in its broader aspects, and, therefore, the aim in the appended claims is to cover all such changes and modifications as fall within the true spirit and scope of the invention.

1. A guidewire for guiding an irrigating tube into and through a body cavity, comprising:
an elongate, resilient shaft adapted to pass in an interior of or along the irrigating tube; and
a bulbous enlargement arranged at a distal end of said shaft, said bulbous enlargement having a smooth, arcuate outer surface.

2. The guidewire of claim 1, wherein said bulbous enlargement is spherical.

3. The guidewire of claim 1, wherein said bulbous enlargement has a diameter of about 0.25 inches to about 0.75 inches.

4. The guidewire of claim 1, where said shaft is formed such that a portion of said shaft attached to said bulbous enlargement is more flexible than the remainder of said shaft.

5. The guidewire of claim 1, wherein said shaft is tapered such that an end attached to said bulbous enlargement has a smaller cross-section than an end distant from said bulbous enlargement.

6. The guidewire of claim 1, wherein said shaft comprises a central metal wire and a coil surrounding said central metal wire.

7. A method for irrigating a body cavity, comprising the steps of:
pushing a guidewire having a bulbous enlargement at a distal end into the body cavity; then
sliding an irrigating tube over or along the guidewire; and then
directing fluid through the irrigating tube.

8. The method of claim 7, wherein the body cavity is the colon, the step of pushing the guidewire into the colon comprising the step of pushing the guidewire through the anal sphincter and rectum into the colon.

9. The method of claim 7, further comprising the step of providing the bulbous enlargement with a smooth outer arcuate surface such that when the guidewire is pushed into the body cavity, contact between the bulbous enlargement and a wall of the body cavity does not cause perforation of the wall of the body cavity.

10. The method of claim 7, further comprising the step of positioning the irrigating tube over the guidewire such that guidewire passes through an interior of the irrigating tube and the irrigating tube slides over the guidewire.

11. The method of claim 7, further comprising the step of guiding the guidewire through the body cavity by manipulating the bulbous enlargement.

12. The method of claim 7, further comprising the steps of:
grasping the bulbous enlargement when present in the body cavity; and then pulling the guidewire outward from the body cavity to cause the body cavity to compress and shorten the distance between an entrance leading to the body cavity and a treatment site at which the bulbous enlargement is positioned.

13. The method of claim 7, further comprising the step of sizing the bulbous enlargement to have a diameter of about 0.25 inches to about 0.75 inches.

14. The method of claim 7, wherein the irrigating tube is slid over the guidewire, further comprising the steps of:
sizing the bulbous enlargement to have a diameter smaller than an inner diameter of the irrigating tube; and
withdrawing the guidewire from the irrigating tube after the irrigating tube is slid over the guidewire and before fluid is directed through the irrigating tube.

15. A colonic irrigation device, comprising:
a guidewire having a bulbous enlargement at a distal end, said guidewire being adapted to be guided into the colon;
an irrigating tube movable relative to and over or along said guidewire; and
means for providing a flow of fluid through said irrigating tube,
whereby said guidewire is guided into the colon and then said irrigating tube is moved over or along said guidewire into the colon.

16. The colonic irrigation device of claim 15, wherein said bulbous enlargement has a smooth, arcuate outer surface.

17. The colonic irrigation device of claim 15, wherein said bulbous enlargement is spherical.

18. The colonic irrigation device of claim 15, wherein said bulbous enlargement has a diameter of about 0.25 inches to about 0.75 inches.

19. The colonic irrigation device of claim 15, wherein said irrigating tube is movable over said guidewire, said bulbous enlargement having a diameter smaller than an inner diameter of said irrigating tube whereby said guidewire is removable from said irrigating tube after said irrigating tube has been slid over said guidewire and before fluid is directed through said irrigating tube.

20. The colonic irrigation device of claim 15, wherein said irrigating tube defines an interior, said guidewire being arranged at least partially in said interior of said irrigating tube such that said irrigating tube slides over said guidewire.

21. The colonic irrigation device of claim 15, wherein said guidewire includes a shaft attached at one end to said bulbous enlargement, said shaft being tapered such that the end attached to said bulbous enlargement has a smaller cross-section than an end distant from said bulbous enlargement.

22. The colonic irrigation device of claim 15, wherein said guidewire includes a shaft attached at one end to said bulbous enlargement, said shaft comprising a central metal wire and a coil surrounding said central metal wire.

23. In a body cavity irrigation device including an irrigating tube through which fluid is directed and a guidewire for guiding the irrigating tube into and through a body cavity, said guidewire including an elongate, resilient shaft adapted to pass in an interior of or along the irrigating tube, the improvement comprising:
said guidewire including a bulbous enlargement arranged at a distal end of said shaft, said bulbous enlargement having a smooth, arcuate outer surface.

24. The irrigation device of claim 23, wherein said bulbous enlargement is spherical.

25. The irrigation device of claim 23, wherein said bulbous enlargement has a diameter of about 0.25 inches to about 0.75 inches.

26. The irrigation device of claim 23, where said shaft is formed such that a portion of said shaft attached to said bulbous enlargement is more flexible than the remainder of said shaft.

27. The irrigation device of claim 23, wherein said shaft is tapered such that an end attached to said bulbous enlargement has a smaller cross-section than an end distant from said bulbous enlargement.

28. The irrigation device of claim 23, wherein said shaft comprises a central metal wire and a coil surrounding said central metal wire.
